(19) United States
(12) Patent Application Publication
(10) Pub. No.: US 2023/0235258 A1
(43) Pub. Date: Jul. 27, 2023

(54) COLONY ANALYSIS APPARATUS FOR DETERMINING CONTAMINATION LEVEL OF FOOD SUBJECT TO MICROBIAL COLLECTION

(71) Applicant: TritonNet Co., Ltd., Seongnam-si (KR)

(72) Inventor: Sung Yong KANG, Yongin-si (KR)

(73) Assignee: TritonNet Co., Ltd., Seongnam-si (KR)

(21) Appl. No.: 18/084,977

(22) Filed: Dec. 20, 2022

(30) Foreign Application Priority Data
Jan. 25, 2022 (KR) 10-2022-0010713

(51) Int. Cl. C12M 1/34; G06T 7/00; C12M 1/22

(52) U.S. Cl. CPC C12M 1/3446; G06T 7/0008; C12M 23/10; G06T 2207/30128

(57) ABSTRACT

Disclosed herein is a colony analysis apparatus, which cultures microorganisms collected from food subject to microbial collection on a plurality of Petri substrates and analyzes colors and the number of the cultured microorganisms by image recognition, thereby accurately determining and analyzing the contamination level of the food subject to microbial collection by an average value of the analysis.

### BACKGROUND OF THE INVENTION

### Field of the Invention

The present invention relates to a colony analysis apparatus, and more particularly, to a colony analysis apparatus, which cultures microorganisms collected from food subject to microbial collection on a plurality of Petri substrates and analyzes colors and the number of the cultured microorganisms by image recognition, thereby accurately determining and analyzing the contamination level of the food subject to microbial collection by an average value of the analysis.

### Background Art

For the purpose of sanitary management of food and medicine, etc., the contamination level is determined by collecting microorganisms from food or medicine, culturing them, and counting the number of the microorganisms.

A microorganism culture sheet is used to culture the collected microorganisms. As an example, Korean Patent No. 10-1811073 discloses a microbial culture sheet, as illustrated in FIG. 1, including: a substrate sheet 10; a dry culture layer 30 formed on the substrate sheet 10, and a cover sheet 40 covering the dry culture layer 30. The cover sheet 40 has an adhesive layer and a release film 45 laminated on the inner surface thereof.

Therefore, the microbial culture sheet can check the contamination level by counting the number of colonies of the microorganisms after the microorganisms are cultured on the dry culture layer 30.

However, as illustrated in FIG. 2, in a case in which the number of the microorganism colonies 50 cultured on the microbial culture sheet, a worker must count them manually while marking count points 60 near the colonies 50 with a pen. Accordingly, the conventional microbial culture sheet is deteriorated in work efficiency and may cause an error in counting.

That is, the contamination level can be accurately figured out when the microorganism colonies 50 are accurately counted. However, since the accuracy of counting is varied according to skills of workers, it is difficult to accurately find the contamination level.

Moreover, since a determination result of the contamination level may be varied according to collection positions of microorganisms and skills of collectors and the contamination level is determined by just one collection of microorganisms, it is difficult to accurately determine the contamination level.

In addition, since the contamination level is determined just by the number of microorganism colonies, in a case in which harmless or unnecessary microorganism colonies are counted, there is high probability of causing an error in determination of the contamination level.

### PATENT LITERATURE

### Patent Documents

Patent Document 1: Korean Patent No. 10-1811073 (Microbial culture sheet)

Patent Document 2: Korean Patent No. 10-0499436 (Microbial culture substrate and medium)

### SUMMARY OF THE INVENTION

Accordingly, the present invention has been made to solve the above-mentioned problems occurring in the prior arts, and it is an object of the present invention to provide a colony analysis apparatus, which cultures microorganisms collected from food subject to microbial collection on a plurality of Petri films and recognizes colors and the number of the cultured microorganisms by image recognition, thereby accurately determining and analyzing the contamination level of the food subject to microbial collection by an average value of the analysis.

To accomplish the above object, according to the present invention, there is provided a colony analysis apparatus including: a petri plate on which a plurality of petri substrates can be placed; a smart phone fixing unit located on an upper side of the Petri plate to fix and hold a smart phone so that a camera of the smart phone can photograph the plurality of Petri substrates, wherein an application is stored in the smart phone to take images of the plurality of Petri substrates by the camera, detect colors and the number of colonies through image recognition, and determine a contamination level.

The colony analysis apparatus further includes: an intake port for inhaling indoor air; a plasma generating unit for generating free radicals to sterilize contaminants floating in the indoor air; an exhaust port for discharging indoor air from which the contaminants are sterilized by the free radicals generated from the plasma generating unit; a lower spray hole formed in the Petri plate below the Petri substrate to spray the free radicals generated from the plasma generating unit; an upper spray hole for spraying the free radicals generated from the plasma generating unit toward the upper surface of the Petri substrate; a Petri sensor for sensing whether or not the Petri substrate is put on the petri plate; and a control unit communicating with the camera, transferring a sensing signal to the smart phone when the Petri sensor senses the Petri substrate, and operating a spray fan when the camera transfers a signal of image acquisition so as to spray the free radicals generated from the plasma generating unit through the lower spray hole and the upper spray hole.

In addition, the application operates to: extract color values from acquired color images of the Petri substrates, extract colonies, and store color location information; extract a chroma value of each colony at a location corresponding to the location information from the color image; set a portion having the highest chroma value among the chroma values of the colonies as a reference point; calculate a distance between the reference point of the colony and the reference point of a neighboring colony; form an outline from the reference point of each colony to an allowable range of the chroma value in a case in which the calculated distance is shorter than a preset distance value; acquire a distance value in a centrifugal manner from the reference point of the colony to the outline to calculate an average distance value; in a case in which the calculated average value is within a recognition range of the colony and the average value is shorter than the distance between the reference point of the colony and the reference point of the neighboring colony, recognize the colonies as one colony and store an intermediate location value; remove noise after converting the color image into an black and white image; divide the black and white image from which noise is removed into a plurality of lattice shapes, and determine the image as a colony if a bitmap matrix in the divided region is more than a preset value but determine the image as noise if a bitmap matrix in the divided region is less than a preset value; calculate the number of colonies by matching the location of the determined colony corresponding to the stored intermediate location value of the colony; and compare the number of the calculated colonies of the plurality of Petri substrates, determine the contamination level according to an average value of the colonies if the number of the colonies is within an error range, and determine as impossible judgment if the number of the colonies is out of the error range.

According to the present invention configured as described above, the colony analysis apparatus can acquire images of a plurality of Petri substrates by using a smart phone, classify colonies having specific colors by the colony recognition module constructed in the smart phone so as to determine the contamination level of food, accurately count the number of the colonies, and accurately determine and analyze the contamination level of food subject to microorganism collection by the average value of the colonies cultured on the plurality of Petri substrates.

Therefore, the colony analysis apparatus can accurately recognize colonies at a low price by using a smart phone so as to enhance determination of the contamination level of food and accuracy in analysis.

In addition, the colony analysis apparatus can prevent spread of contamination by removing contaminants by spraying free radicals to the Petri substrates.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a view illustrating a structure of a conventional microbial culture sheet;

FIG. 2 is a photograph showing a state in which the number of colonies cultured on a microbial culture sheet is counted manually;

FIG. 3 is a view illustrating an external structure of a colony analysis apparatus according to an embodiment of the present invention;

FIG. 4 is a view illustrating a cross-sectional structure of FIG. 3;

FIGS. 5 to 14 are views illustrating a process of recognizing colonies by a smartphone application;

FIG. 15 is a view showing an example of a screen displaying a colony count result recognized from the smartphone; and

FIG. 16 is a circuit block diagram of a body and the smartphone.

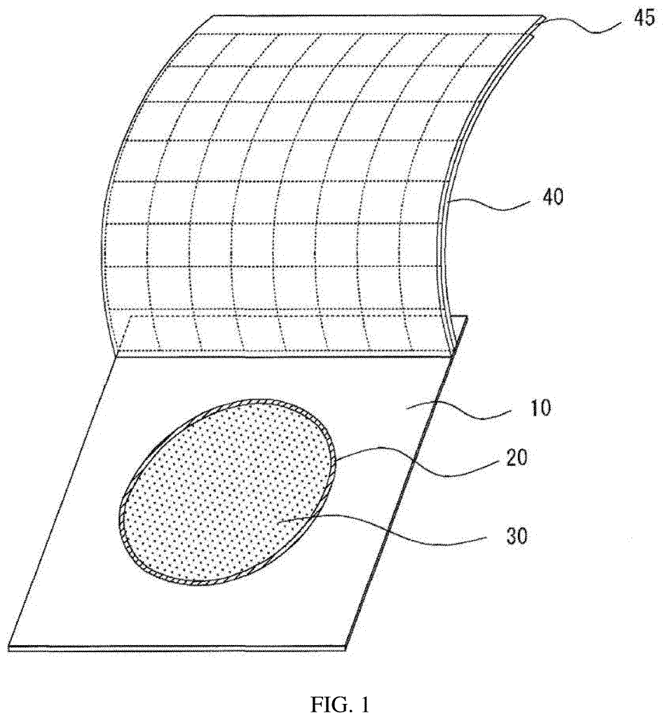

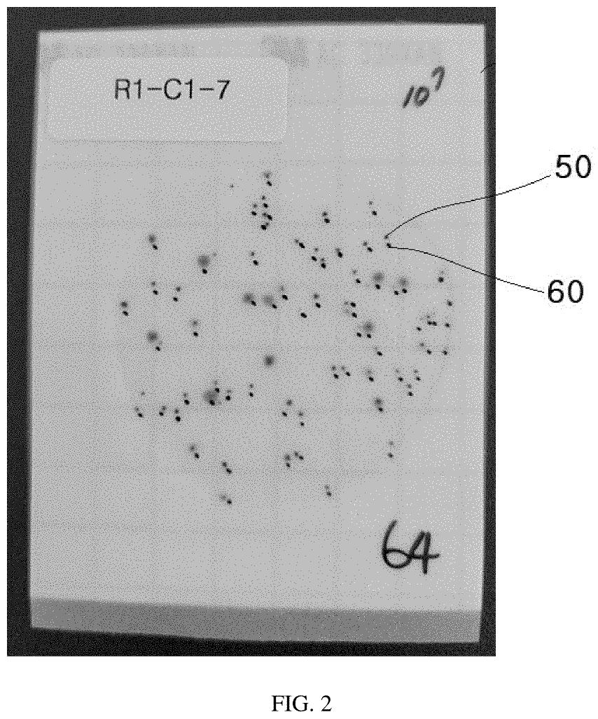

### DETAILED DESCRIPTION OF THE PREFERRED EMBODIMENT

The above-described objects, features and advantages will be described below in detail with reference to the accompanying drawings, and accordingly, those skilled in the art to which the present invention pertains will be able to easily implement the technical idea of the present invention.

In description of the present invention, when it is judged that detailed descriptions of known functions or structures related with the present invention may make the essential points vague, the detailed descriptions of the known functions or structures will be omitted.

Hereinafter, embodiments of the present invention will be described in detail with reference to the accompanying drawings.

However, the embodiments of the present invention illustrated below may be modified in various other forms, and the scope of the present invention is not limited to the embodiments described below.

The embodiments of the present invention are provided to more completely explain the present invention to those of ordinary skill in the art.

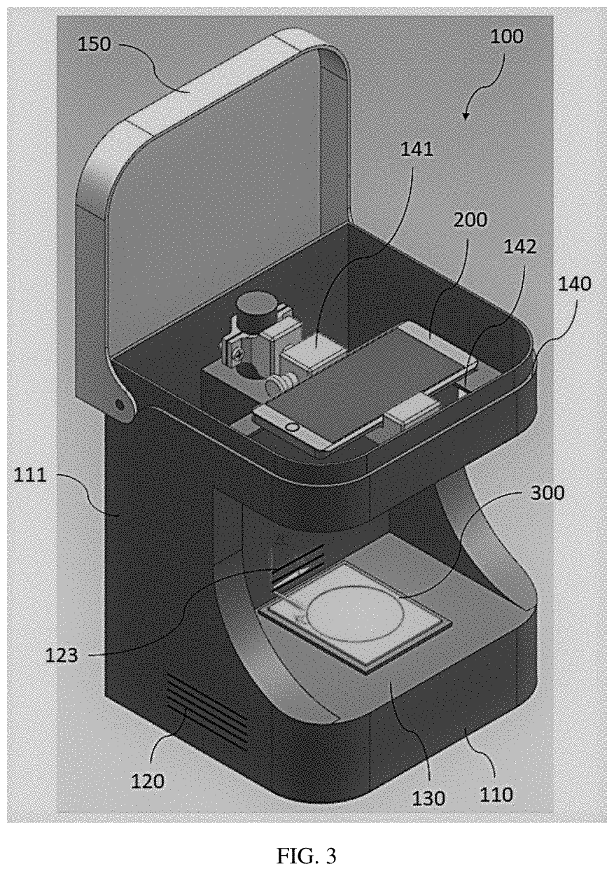

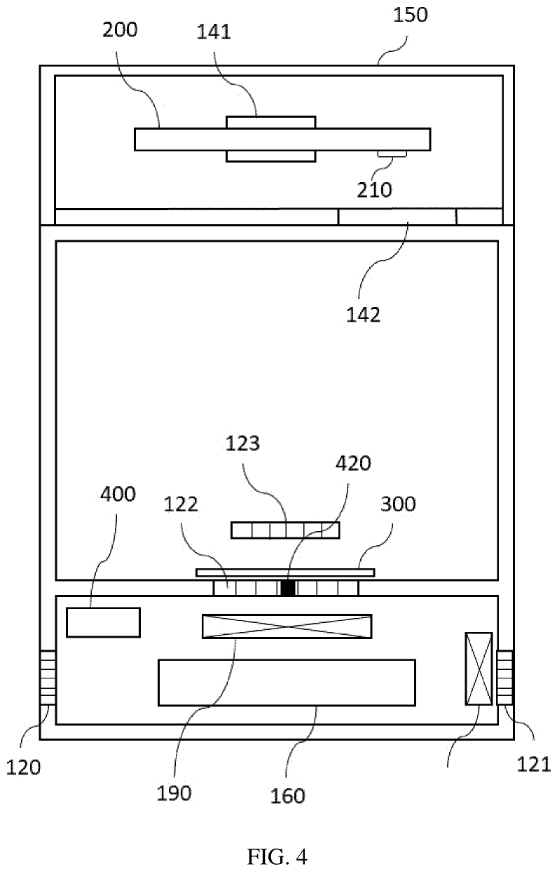

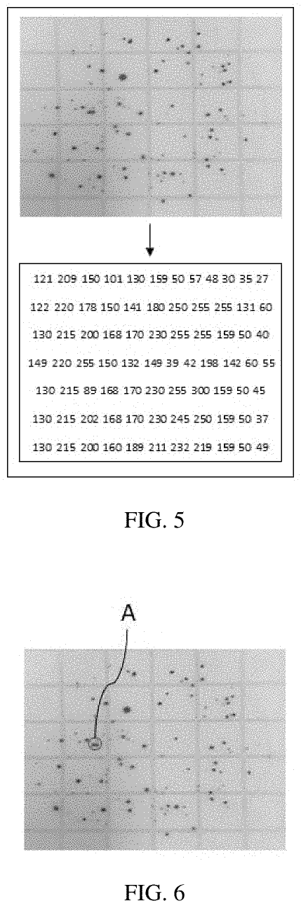

FIG. 3 is a view illustrating an external structure of a colony analysis apparatus according to an embodiment of the present invention, FIG. 4 is a view illustrating a cross-sectional structure of FIG. 3, and FIGS. 5 to 14 are views illustrating a process of recognizing colonies by a smartphone application.

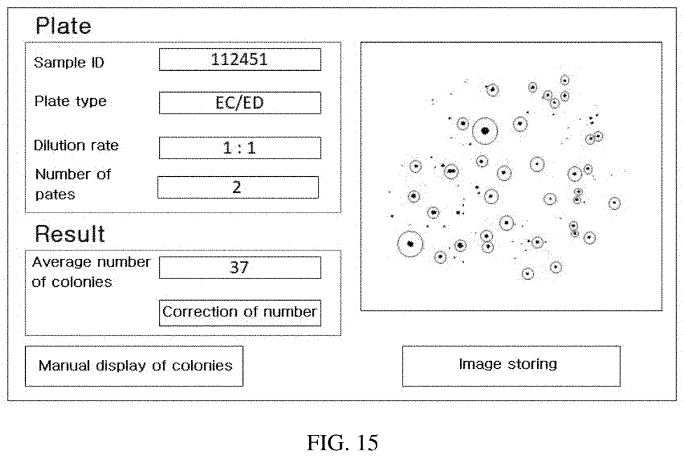

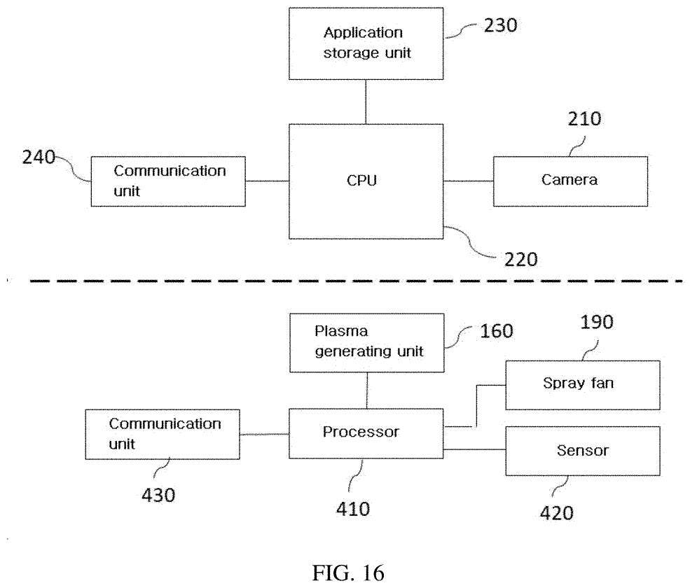

Furthermore, FIG. 15 is a view showing an example of a screen displaying a colony count result recognized from the smartphone, and FIG. 16 is a circuit block diagram of a body and the smartphone.

As illustrated in FIG. 3, the colony analysis apparatus 100 according to the present invention includes a body 110, and a Petri plate 130 disposed on the upper surface of the body 110 for placing a Petri substrate 300 of a film material on which germs, bacteria, viruses, etc. are cultured. The body 110 includes an upper extension 111 formed at the rear portion thereof to extend upward.

A smartphone fixing unit 140 for fixing a smartphone 200 is formed integrally with an upper portion of the upper extension 111 to protrude forward. A lateral cross section of a sterilizer 100 is formed in an approximately [symbol] shape.

The smartphone fixing unit 140 has a fixing arm 140 for elastically gripping and supporting the smartphone 200 at both sides, and the fixing arm 140 has a structure capable of rotating in a horizontal direction and in a vertical direction.

Since such a structure is general in the smartphone holder field, a detailed description of the smartphone fixing unit 140 will be omitted.

A cover 150 is mounted on the smartphone fixing unit 140 in a vertically rotatable manner so as to prevent external contaminants from being introduced into the smartphone fixing unit 140 and prevent the smartphone 200 from being contaminated.

The cover 150 is preferably formed of a transparent material, and in the case of a transparent material, it is possible to visually check the operating state of the smartphone 200.

The smartphone fixing unit 140 has an exposure hole 142 formed at the bottom, so that a camera 210 of the smartphone 200 can take a picture of the Petri substrate 300 through the exposure hole 142 when the camera 210 of the smartphone 200 is fixed to a fixing arm 141 of the smartphone fixing unit 140 to face downward. When a grip position and a rotational state of the fixing arm 141 is adjusted, the camera 210 of the smartphone 200 can more accurately photograph the Petri substrate 300.

Once the smartphone 200 is fixed to the smartphone fixing unit 140, the cover 150 may be closed to prevent contamination while photographing a plurality of petri substrates 300.

The smartphone 200 is provided with the camera 210 for acquiring an image by photographing the Petri substrate 300. The camera 210 currently has tens of millions to 100 million or more pixels, so it has very high resolution. Additionally, a portable camera may be mounted on the smartphone, so the apparatus may be implemented at a very cheap price.

The smartphone 200 includes an application storage unit 230 disposed therein to store an application for counting colonies cultured on the Petri substrate 300 by image recognition, and a colony recognition module is constructed in the application by machine learning.

Referring to FIGS. 5 to 15, the process of recognizing and counting colonies by the colony recognition module will be described. As illustrated in FIG. 4, a color image of the Petri substrate 300 photographed by the camera 210 is acquired from a CPU 220, and then, a color value is acquired by the application.

The reason is to recognize only microorganism colonies having a specific color, for instance, red, blue, or the like, in order to determine the contamination level.

Accordingly, as illustrated in FIG. 5, pixels of an image having a specific color, for example, a colony having a red color (expressed in black in the drawing) and a petri substrate of a white color, are converted into digital value, and only a value of the red color or a value close to the red color is extracted from the digital values to separate specific colonies. The color location information is stored in the application storage unit 230.

In addition, a chroma value of each colony is acquired from the color image of the photographed Petri substrate 300. In this instance, in a case in which the plurality of colonies cultured on the Petri substrate 300 are spaced apart from one another at predetermined intervals, the colonies may be counted one by one. However, in a case in which a colony is very close to a neighboring colony, the colonies are recognized as one colony. Accordingly, the colonies are separated through pre-treatment to make it possible to count accurately.

That is, specific colonies are separated by color values, and overlapping colonies are accurately separated by chroma values to be recognized accurately.

So, the chroma value is extracted from a location corresponding to the color location information stored in the application storage unit 230 from the color image of the Petri substrate 300. FIGS. 6 to 10 illustrate the process of separating two adjacent colonies by the chroma value for convenience of description.

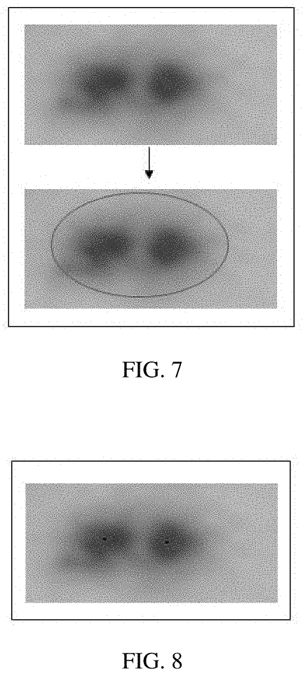

The color image of the Petri substrate of FIG. 6 is scanned to extract the chroma value for each pixel. FIG. 7 is an enlarged view of the red circle part (A) of FIG. 6 illustrating a state in which neighboring colonies are very close to each other.

In a case in which the neighboring colonies overlap each other while the microorganism colonies cultured on the Petri substrate 300 are growing during the cultivation process, the neighboring colonies may be determined as one colony. Accordingly, it is preferable to separate and recognize the neighboring colonies.

Therefore, the chroma value is checked from the color image as in FIG. 8, and a region with the highest chroma value is set as the reference point. In a case in which the plurality of colonies are close to each other, a distance between the reference points is very short, and the CPU 220 calculates a distance value between the reference points.

In a case in which the calculated distance value is smaller than a preset distance value, an outline is formed from the reference point to an allowable range of the chroma value. In this instance, in a case in which the calculated distance value is larger than the preset distance value, the colonies are easily separated by the image recognition to promote counting.

That is, since an outer region of one colony does not overlap with an outer region of the neighboring colony, they can be easily separated, thereby making counting easier.

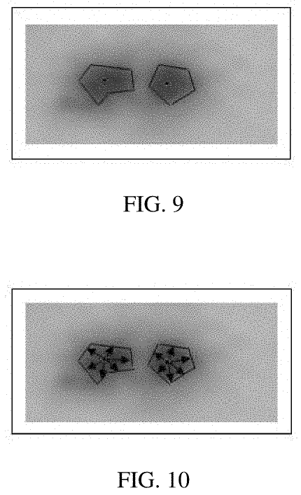

However, in a case in which the reference point of one colony is close to the reference point of the neighboring colony and the distance value is smaller than the preset distance value, the outer regions of the two neighboring colonies overlap each other so that the colonies are determined as one colony. Accordingly, the neighboring colonies must be separated from each other. For this purpose, as illustrated in FIG. 9, an outline is formed up to the range allowed by the chroma value.

That is, the outline is formed up to an allowable range having a ½ or ⅓ chroma value of the chroma value of the reference point, excluding a region which has a low chroma value due to a long distance from the reference point.

The allowable range of the chroma values is previously set as a value that does not overlap with the allowable range of the chroma value of the neighboring colony.

Thereafter, as illustrated in FIG. 10, the distance value is obtained in a centrifugal manner from the reference point of the colony to the outline to measure an average value. In a case in which the average value is a value for the recognition range of the microorganism colony, that is, a value corresponding to the size of the colony cultured for a certain period of time rather than noise, information of the location is stored in the application storage unit 230.

In a case in which the distance value is short of the average distance value even though the chroma value of the reference point is high, there is a high possibility not the cultured microorganism colonies but other foreign matters or noises are recognized. Accordingly, the average distance value is measured to accurately count only the colonies to maximize accuracy in counting.

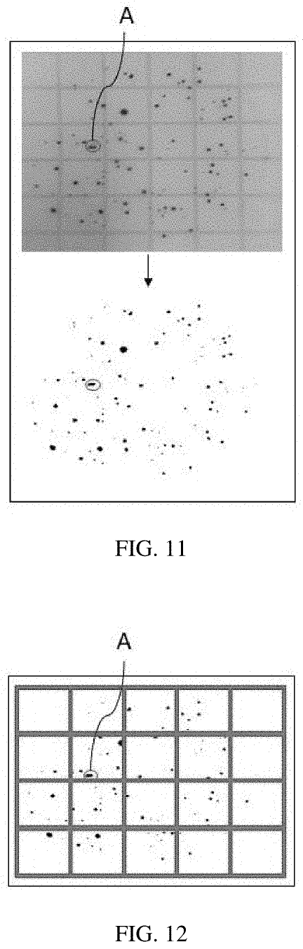

Thereafter, as illustrated in FIG. 11, the color image of FIG. 6 is converted into a black-and-white image, and a binary value having 0 for white and 255 for black is provided.

Noise is removed from the image converted into black and white through an image thresholding process. A clear image made in white and black can be output when the noise of the background color is removed.

That is, black colonies can be accurately extracted by clearly separating white and black based on the threshold value so that modeling for image recognition can be performed accurately.

The image from which the noise is removed has a clear boundary of a black color corresponding to the colony, so that a worker can perform modeling work easily.

Thereafter, as illustrated in FIG. 12, the black and white image from which noise is removed is divided into several regions having a grid shape. Such image division is very advantageous in terms of precision while the higher the division density, the higher the image processing capacity. Since the smartphone currently used are very fast in processing speed and are large in processing capacity, image processing can be performed with no difficulty.

Each divided region distinguishes a colony from noise by a pixel-unit bitmap matrix. For example, if 15 or more values set to 225 and consecutively arranged form a bitmap matrix, it is determined as a colony, and conversely, if 15 or less values set to 225 form a bitmap matrix, it is determined as noise.

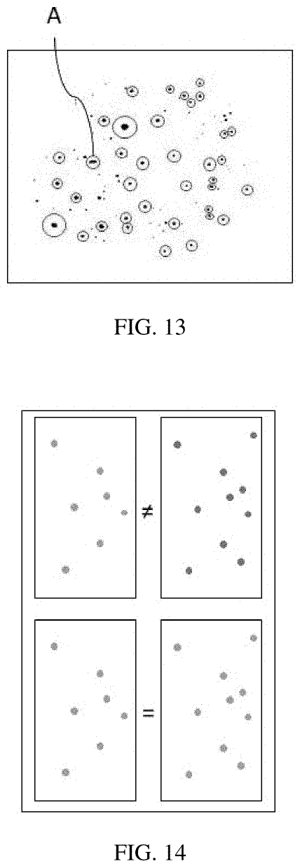

Furthermore, as illustrated in FIG. 13, a red circle may be expressed around the extracted colony to visually express the colony recognition result.

In this instance, although the ‘A’ part is recognized and displayed as one colony, since the position values recognizing as two colonies through the pre-treatment of the color image has been stored, the ‘A’ part is divided and recognized as two colonies by matching the location of the colony corresponding to the stored colony location values. Therefore, the ‘A’ part is counted as two colonies.

Microorganisms collected from the same microbial collection target food are cultured on a plurality of Petri substrates 300, and then, colonies of each Petri substrate are counted. A photographed color image and colony information extracted from the color image, namely, colors of the colonies, the number of the colonies, and the like, are stored.

An average value is obtained in comparison with the colony information extracted from the color images of the Petri substrates 300, and the contamination level is accurately determined by the average value. First, as illustrated in FIG. 14, the images of the colonies are compared with each other. That is, the image of the colony having a specific color, for instance, a red color, (shown at the left side of the drawing) is compared with the image of another colony having a different color (shown at the right side of the drawing) in order to determine the contamination level. In a case in which the color value of the another colony is a blue color, the image of the another colony is excluded, and only the image of the colony having the same red color is selected, and a stored colony count value of each image of the colony is read.

Accordingly, the contamination level is determined by the average value of the colony count value read from the plurality of colony images. In a case in which a colony count value of a specific colony image is higher or lower than the average value, the colony count value of the corresponding colony image is excluded, and then, the average value is recalculated to extract a more accurate average colony value.

That is, in a case in which the colony count value is deviated from an allowable range, since there is an error in collection of microorganisms or there is a possibility that different microorganisms are collected, the colony count value is excluded to determine the contamination level more accurately.

Therefore, the average value of the recognized colonies may be displayed on a screen of the smart phone 200 or may be transferred to a computer through a communication unit 240 to be displayed on a screen of the computer as illustrated in FIG. 16.

FIG. 15 illustrates a state in which a button for correcting a sample name, a Petri type, a dilution rate, the number of plates, the average number of colonies, and the number of colonies counted manually, a button for manually displaying colonies while watching a displayed colony count image, and a button for storing a final image are displayed on a monitor screen.

Meanwhile, the body 110 includes a plasma generating unit 160 mounted therein to generate free radicals, and an intake port 120 and an exhaust port 121 respectively formed at the left and right sides of the body. Indoor air is sucked to the intake port 120 through an exhaust fan 170 to react and sterilize free radicals and contaminants, and then, is exhausted indoors through the exhaust port 121.

Since technique to sterilize germs, bacteria, and viruses which are indoor contaminants by the plasma have been mentioned in the conventional arts and is a general matter, a detailed description thereof will be omitted.

The Petri plate 130 has a lower spray hole 122 of a mesh or lattice-shape on which the Petri substrate 300 is put, and an upper spray hole 123 formed on an inner surface of an upper extension portion 111, namely, a forward exposure portion to face the upper surface of the Petri substrate 300.

Therefore, The free radicals generated from the plasma generating unit 160 are sprayed toward the upper surface and the lower surface of the Petri substrate 300 put on the lower spray hole 122. The Free radicals are supplied to the lower spray hole 122 and the upper spray hole 123 by a spray fan 190 so as to sterilize contaminants, such as germs, bacteria, viruses and the likes, existing on the outer surface of the Petri substrate 300.

Operations of the plasma generating unit 160, the exhaust fan 170, and the spray fan 190 are controlled by a control unit 400 disposed inside the body 110. A Petri sensor 420 is disposed at the position of the lower spray hole 122 to sense whether or not the Petri substrate 300 is put on the lower spray hole.

The Petri sensor 420 may be one of various sensors, such as an optical sensor, a capacitance sensor, or the like. The control unit 400 operates the spray fan 190 according to a sensing result of the Petri sensor 420 so as to spray the free radicals to the upper surface and the lower surface of the Petri substrate 300. In a normal operation, the control unit 400 operates the exhaust fan 170 to inhale and sterilize indoor air, but when the Petri substrate 300 is sensed, the control unit 400 operates also the spray fan 190 to sterilize contaminants stained on the surface of the Petri substrate 300.

In addition, the control unit 400 and the smartphone 200 may interwork with each other so as to automatically perform photographing of the Petri substrate 300 and sterilization of the Petri substrate 300.

Accordingly, the control unit 400 includes a communication unit 430 for shortrange communication, such as Bluetooth, with the smart phone 200. When the Petri substrate 300 is put on the Petri plate 130, the Petri sensor 420 senses the Petri substrate 300, and according to the sensing result, the processor 410 transfers a sensing signal to the smart phone 200 through the communication unit 430.

The CPU 220 of the smart phone 200 operates the camera 210 by the sensing signal input through the communication unit 240 to obtain an image of the Petri substrate 300. Thereafter, the application stored in the application storage unit 230 counts colonies and displays the counted result or transfers it to the computer.

Thereafter, when the smart phone 200 transfers a signal of image acquisition to the control unit 400, the processor 410 receives the signal through the communication unit 430 and operates the spray fan 190, so that free radicals are sprayed to the Petri substrate 300 through the lower spray hole 122 and the upper spray hole 123 to perform sterilization.

As described above, while the present invention has been particularly shown and described with reference to the example embodiments thereof, it will be understood by those of ordinary skill in the art that the above embodiments of the present invention are just exemplified, and the scope of the present invention is not limited thereby.

Accordingly, it is intended that the substantial scope of the present invention be defined by the appended claims and their equivalents.

What is claimed is:

1. A colony analysis apparatus comprising:
a petri plate on which a plurality of petri substrates can be placed;
a smart phone fixing unit located on an upper side of the Petri plate to fix and hold a smart phone so that a camera of the smart phone can photograph the plurality of Petri substrates,
wherein an application is stored in the smart phone to take images of the plurality of Petri substrates by the camera, detect colors and the number of colonies through image recognition, and determine a contamination level.

2. The colony analysis apparatus according to claim 1, wherein the application operates to:
extract color values from acquired color images of the Petri substrates, extract colonies, and store color location information;
extract a chroma value of each colony at a location corresponding to the location information from the color image;
set a portion having the highest chroma value among the chroma values of the colonies as a reference point;
calculate a distance between the reference point of the colony and the reference point of a neighboring colony;
form an outline from the reference point of each colony to an allowable range of the chroma value in a case in which the calculated distance is shorter than a preset distance value;
acquire a distance value in a centrifugal manner from the reference point of the colony to the outline to calculate an average distance value;
in a case in which the calculated average value is within a recognition range of the colony and the average value is shorter than the distance between the reference point of the colony and the reference point of the neighboring colony, recognize the colonies as one colony and store an intermediate location value;
remove noise after converting the color image into a black and white image;
divide the black and white image from which noise is removed into a plurality of lattice shapes, and determine the image as a colony if a bitmap matrix in the divided region is more than a preset value but determine the image as noise if a bitmap matrix in the divided region is less than a preset value;
calculate the number of colonies by matching the location of the determined colony corresponding to the stored intermediate location value of the colony; and
compare the number of the calculated colonies of the plurality of Petri substrates, determine the contamination level according to an average value of the colonies if the number of the colonies is within an error range, and determine as impossible judgment if the number of the colonies is out of the error range.

3. The colony analysis apparatus according to claim 1, further comprising:
an intake port for inhaling indoor air;
a plasma generating unit for generating free radicals to sterilize contaminants floating in the indoor air;
an exhaust port for discharging indoor air from which the contaminants are sterilized by the free radicals generated from the plasma generating unit;
a lower spray hole formed in the Petri plate below the Petri substrate to spray the free radicals generated from the plasma generating unit;
an upper spray hole for spraying the free radicals generated from the plasma generating unit toward the upper surface of the Petri substrate;
a Petri sensor for sensing whether or not the Petri substrate is put on the petri plate; and
a control unit communicating with the camera, transferring a sensing signal to the smart phone when the Petri sensor senses the Petri substrate, and operating a spray fan when the camera transfers a signal of image acquisition so as to spray the free radicals generated from the plasma generating unit through the lower spray hole and the upper spray hole.
